(19) United States
(12) Patent Application Publication
(10) Pub. No.: US 2026/0036575 A1
(43) Pub. Date: Feb. 5, 2026

(54) DEVELOPMENT OF BIOLAYER INTERFEROMETRY (BLI) BASED DOUBLE STRAND RNA DETECTION UTILIZING FHV B2 PROTEIN

(71) Applicant: Regeneron Pharmaceuticals, Inc., Tarrytown, NY (US)

(72) Inventors: Yue FU, Pleasantville, NY (US); Dharia Sara SILAS, Bronx, NY (US); Bindiya JUNEJA, Harrison, NY (US); Kathir MUTHUSAMY, Cheshire, CT (US); Nisha PALACKAL, White Plains, NY (US); Erica PYLES, New City, NY (US)

(73) Assignee: Regeneron Pharmaceuticals, Inc., Tarrytown, NY (US)

(21) Appl. No.: 19/284,944

(22) Filed: Jul. 30, 2025

Related U.S. Application Data
(60) Provisional application No. 63/677,147, filed on Jul. 30, 2024.

(51) Int. Cl. G01N 33/53; G01N 33/543

(52) U.S. Cl. CPC G01N 33/5308; G01N 33/54306; G01N 2333/08

(57) ABSTRACT

The present invention provides methods and systems to identify and quantify double-stranded RNA (dsRNA) in a sample. A capture molecule can be immobilized to a solid surface. The solid surface can then be contacted with a sample including the dsRNA, wherein the capture molecule immobilized on the solid surface specifically binds to the dsRNA. The binding response of the dsRNA to the solid surface can then be measured by biolayer interferometry to detect the dsRNA in the sample. The measured binding response can be compared to a standard curve relating binding response to concentration to determine a concentration of the dsRNA in the sample.

### CROSS-REFERENCE TO RELATED APPLICATION

This application claims priority to U.S. Provisional Patent Application No. 63/677,147, filed on Jul. 30, 2024, which is hereby incorporated by reference in its entirety.

### SEQUENCE LISTING

This application contains a sequence listing, submitted electronically in XML format and incorporated by reference herein in its entirety. The XML copy of the sequence listing, with the filename Sequence_Listing.xml, was created on Jul. 28, 2025, and is 2,920 bytes in size.

### INTRODUCTION

mRNA-based therapeutics have emerged as a rapidly growing class of therapeutic drugs for the treatment of infectious diseases. The success of mRNA-based therapeutics has highlighted the need for robust analytical methods to determine safety, potency, and purity. mRNA products are often synthesized by in vitro transcription, whereby a DNA template is transcribed into the corresponding mRNA using an RNA polymerase. mRNA synthesis by in vitro transcription can result in unwanted side products, such as double-stranded RNA. It has been demonstrated that dsRNA can strongly interfere with protein translation and activate innate immunity. Thus, this impurity has the potential to affect the potency and safety of mRNA drug products.

Analytical methods for characterizing dsRNA in mRNA products are needed during the manufacturing process. Several analytical methods are commonly used to detect and quantify dsRNA, including gel electrophoresis, enzyme-linked immunosorbent assay (ELISA), or homogeneous time resolve fluorescence (HTRF), which have low sensitivity or are too time consuming to perform. Recent reports have also demonstrated the use of lateral flow immunoassay (LFSA) for dsRNA quantitation, which requires less time to run but lacks the sensitivity for dsRNA with modified uridine. One possible explanation for the reduced sensitivity in these methods is that these techniques rely on the use of anti-dsRNA antibodies, which have been demonstrated to have reduced sensitivity for modified dsRNA.

Therefore, it will be appreciated that there is a need for methods and systems to monitor dsRNA levels in RNA products that can overcome the challenges associated with conventional dsRNA detection.

### SUMMARY

Aspects of the present disclosure may include a method for detecting double-stranded RNA (dsRNA) in a sample. The method may include immobilizing a capture molecule to a solid support, contacting a sample including dsRNA to the solid support, and/or measuring a binding response of the dsRNA to the capture molecule using biolayer interferometry. The capture molecule may specifically bind to dsRNA.

Aspects of the present disclosure may include a method for quantifying dsRNA in a sample. The method may include immobilizing a capture molecule to a solid support, contacting a sample including dsRNA to the solid support, measuring a binding response of the dsRNA to the capture molecule using biolayer interferometry, and/or comparing the binding response to a standard curve to determine a concentration of the dsRNA in the sample. The capture molecule may bind to dsRNA. The standard curve may relate binding response to concentration.

Aspects of the present disclosure may include where the dsRNA comprises a modified nucleoside. The dsRNA may comprise pseudouridine, N1-methylpseudouridine, and/or 5-methoxyuridine. The dsRNA may comprise a duplex structure or a hairpin loop. The dsRNA may be present in the sample at a concentration of about 5 ng/mL to about 5000 ng/mL. A concentration of double-stranded DNA (dsDNA) in the sample is greater than a concentration of dsRNA in the sample and/or a concentration of single-stranded RNA (ssRNA) in the sample is greater than a concentration of dsRNA in the sample. The capture molecule may be an antibody, a receptor, an antibody fragment, a receptor fragment, or a combination thereof. The capture molecule may include a J2 antibody, a K1 antibody, a J5 antibody, a Flock House Virus (FHV) B2 protein, or a combination thereof. Immobilizing the capture molecule to the solid support may comprise contacting a biotinylated capture molecule to a surface of the solid support, where the surface comprises avidin, streptavidin, or a variant thereof. Immobilizing the capture molecule to the solid support may comprise immobilizing the capture molecule at a density of at least about 4 molecules per square nanometer. The sample may be a product of mRNA in vitro transcription. The sample may further comprise dsDNA and/or ssRNA. A concentration of dsDNA in the sample may be more than 100-fold greater than the concentration of dsRNA in the sample, and/or a concentration of ssRNA in the sample may be more than 100-fold greater than the concentration of dsRNA in the sample. Contacting the sample to the solid support may cause a dsRNA molecule within the sample to bind to one or more capture molecules. The standard curve may be fit to a 4-parameter logistic model. Methods described herein may further include, after immobilizing the capture molecule and prior to contacting the sample to the solid support, washing the solid support, thereby removing unbound capture molecule.

### BRIEF DESCRIPTION OF THE DRAWINGS

The accompanying drawings, which are incorporated in and constitute a part of this specification, illustrate various exemplary embodiments, and together with the description, serve to explain the principles of the disclosed embodiments. Any features of an embodiment or example described herein (e.g., composition, formulation, method, etc.) may be combined with any other embodiment or example, and all such combinations are encompassed by the present disclosure. Moreover, the described systems and methods are neither limited to any single aspect nor embodiment thereof, nor to any combinations or permutations of such aspects and embodiments. For the sake of brevity, certain permutations and combinations are not discussed and/or illustrated separately herein.

FIG. 1 shows a schematic illustration of the BLI dsRNA detection assay, comprising: (1) mRNA sample dilution, (2) BLI signal readout, and (3) dsRNA detection and quantitation, according to aspects of the present disclosure.

FIG. 2 shows a schematic illustration of the bio-layer interferometry (BLI) method for measuring binding affinity between Flock House Virus (FHV) B2 protein and a double-stranded RNA (dsRNA) with a representative sensorgram, according to aspects of the present disclosure.

FIG. 3 shows binding sensorgrams (top) and resulting binding affinity curves (bottom) for FHV B2 binding to 700 bp dsRNA with (left to right) uridine (U), pseudouridine (W), N1-methylpseudouridine (m1ψ), or 5-methoxyuridine (5moU), according to aspects of the present disclosure.

FIG. 4 shows a schematic illustration of the BLI dsRNA detection assay for detecting B2 binding to dsRNA with a representative sensorgram, according to aspects of the present disclosure.

FIG. 5A shows dsRNA detection by BLI for three in vitro transcription (IVT) mRNA samples, mRNA-1, mRNA-2, and mRNA-3, according to aspects of the present disclosure.

FIG. 5B shows dsRNA detection by HTRF for three in vitro transcription (IVT) mRNA samples, mRNA-1, mRNA-2, and mRNA-3, according to aspects of the present disclosure.

FIGS. 6A, 6B, and 6C show specificity and interference testing of the BLI dsRNA detection assay using chemically similar nucleic acids, according to aspects of the present disclosure. FIG. 6A shows specificity testing: BLI signal is shown for 700 bp-U, 700 bp-mly, ssRNA (100-fold), dsDNA (100-fold), 142 bp dsRNA and Poly(I:C); FIG. 6B shows interference testing: BLI signal of 700 bp-U compared against 700 bp-U in the presence of 100-fold or 200-fold excess of ssRNA or dsDNA; FIG. 6C shows interference testing: BLI signal of 700 bp-U was compared against 700 bp-m1ψ in the presence of 100-fold or 200-fold excess of ssRNA or dsDNA. All experiments were performed in triplicate.

FIGS. 7A, 7B, and 7C show overlays of standard curves of 700 bp dsRNA with different modifications, U, ψ, m1ψ, and 5moU, obtained from BLI (FIG. 7A), HTRF assay (FIG. 7B), and J2 ELISA (FIG. 7C), according to aspects of the present disclosure.

FIG. 8 shows standard curves of BLI signal of FHV B2, J2 antibody, and K1 antibody binding to 700 bp-mly, according to aspects of the present disclosure.

FIGS. 9A and 9B show the standard curve of BLI signal of FHV B2 binding to 700 bp-m1ψ in the presence and absence of excess single-stranded RNA (ssRNA; dsRNA:ssRNA ratio of 1:2.5), according to aspects of the present disclosure.

FIGS. 10A, 10B, and 10C show overlays of standard curves of dsRNA of different lengths, 25 bp, 700, bp, 1800 bp, and hairpin, obtained from BLI (FIG. 10A), HTRF assay (FIG. 10B), and J2 ELISA (FIG. 10C), according to aspects of the present disclosure.

FIGS. 11A and 11B show standard curves of BLI signals of FHV B2 binding to dsRNAs of different lengths, 1800 bp, 25 bp, and hairpin, and different uridine modifications, U and m1ψ, according to aspects of the present disclosure.

FIG. 12 shows detection of dsRNA in two IVT mRNA samples, mRNA-4 and mRNA-5, by BLI (top) and HTRF (bottom) before and after purification by ion-paired reverse phase chromatography, according to aspects of the present disclosure.

### DETAILED DESCRIPTION

Unless defined otherwise, all technical and scientific terms used herein have the same meaning as is commonly understood by one of ordinary skill in the art to which this disclosure belongs. Although any suitable methods and materials (e.g., similar or equivalent to those described herein) can be used in the practice or testing of the present disclosure, particular example methods are now described. All publications mentioned are hereby incorporated by reference.

As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” For the terms “for example” and “such as,” and grammatical equivalences thereof, the phrase “and without limitation” is understood to follow unless explicitly stated otherwise.

As used herein, the term “about” is meant to account for variations due to experimental error. When applied to numeric values, the term “about” may indicate a variation of +/−5% from the disclosed numeric value, unless a different variation is specified. As used herein, the singular forms “a,” “an,” and “the” include plural referents unless the context clearly dictates otherwise. Further, all ranges are understood to be inclusive of endpoints, e.g., from 1 centimeter (cm) to 5 cm would include lengths of 1 cm, 5 cm, and all distances between 1 cm and 5 cm.

It should be noted that all numeric values disclosed or claimed herein (including all disclosed values, limits, and ranges) may have a variation of +/−5% from the disclosed numeric value unless a different variation is specified.

As used herein, the term “sample” refers to a mixture of molecules that comprises at least one dsRNA that is subjected to manipulation in accordance with the methods of the invention, including, for example, separating, analyzing, extracting, concentrating, profiling and the like.

As used herein, the terms “nucleic acid,” “polynucleotide,” or “oligonucleotide” refer a polymer composed of nucleotides or nucleosides (including ribonucleotides, deoxyribonucleotides, or analogs or modified versions thereof), which have nitrogenous heterocyclic bases or base analogs linked together along a backbone. Oligonucleotides may be isolated from genes, or chemically synthesized by methods known in the art. A nucleic acid “backbone” may be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid may be ribose, deoxyribose, or similar compounds with optional substitutions, e.g., methoxy or 2′ halide substitutions. Oligonucleotides may be DNA (deoxyribonucleic acid), RNA (ribonucleic acid), or a hybrid. Oligonucleotides may be single-stranded DNA (ssDNA), single-stranded RNA (ssRNA), double-stranded RNA (dsRNA), double-stranded DNA (dsDNA), or double-stranded DNA/RNA hybrids. Oligonucleotides may be of a variety of different lengths, depending on the form. Oligonucleotides often range in size from a few monomeric units, e.g. 3-4, to hundreds of monomeric units. Whenever an oligonucleotide is represented by a sequence of letters, such as “ATGCCTG,” it will be understood that the nucleotides are in 5′→3′ order from left to right and that “A” denotes adenosine, “C” denotes cytosine, “G” denotes guanosine, and “T” denotes thymidine, unless otherwise noted. The letters A, C, G, and T may be used to refer to the bases themselves, to nucleosides, or to nucleotides comprising the bases, as is standard in the art.

The term “DNA” or “DNA molecule” refers to a chain of nucleotides comprising deoxyribonucleotides that each comprise one of four nucleobases, namely, adenine (A), thymine (T), cytosine (C), and guanine (G). The term “RNA” or “RNA molecule” refers to a chain of nucleotides comprising four types of ribonucleotides that each comprise one of four nucleobases, namely; A, uracil (U), G, and C. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G).

In one exemplary embodiment, the RNA molecule is a mRNA molecule. The term “mRNA” refers to messenger RNA. The term “messenger RNA (mRNA)” refers to a type of nucleic acid, more specifically a single stranded RNA molecule that is synthesized during transcription, is complementary to one of the strands of double-stranded DNA, and serves to transmit the genetic information contained in DNA to the ribosomes for protein synthesis. The mRNA may be spliced, partially spliced or unspliced, and may be eukaryotic or prokaryotic mRNA. The mRNA may also be produced via de novo synthesis.

In an exemplary embodiment, the mRNA is produced by in vitro transcription (IVT) of a DNA molecule encoding the mRNA to be synthesized. The DNA encoding the RNA to be synthesized contains an RNA polymerase promoter to which RNA polymerase binds and initiates transcription. In one aspect, the RNA polymerase is a T7 RNA polymerase. The IVT process may produce nucleic acid byproducts, such as for example, long or short dsRNA, dsDNA or ssRNA.

Oligonucleotides may be modified, e.g., comprise a modified nucleotide, a modified internucleoside linkage, and/or a modified sugar moiety, or combinations thereof. In some embodiments, particular nucleotide modification(s) may be incorporated that render the oligonucleotide more therapeutically effective. In an exemplary embodiment, the modification is a uridine modification, such as, for example, pseudouridine (W), N1-methylpseudouridine (m1ψ), or 5-methoxyuridine (5moU).

As used herein, the term “capture molecule” refers to any molecule that may be used to capture or bind an oligonucleotide or nucleic acid of interest. A capture molecule may be immobilized to a solid surface, for example, through a biotin-streptavidin interaction. A capture molecule may be a target molecule for a dsRNA. For example, a capture molecule may be a protein, such as an dsRNA binding protein. A capture molecule may also be an antibody, a receptor, a fragment thereof, or a combination thereof. One or more suitable capture molecules may be used to capture the oligonucleotide or nucleic acid of interest. In one exemplary aspect, the capture molecule may capture one or more nucleic acid of interest.

As used herein, the term “protein” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule.

In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Biotechnology and Genetic Engineering reviews, 2012, 28, 147-176, the entirety of which is herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the present invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” (bsAbs) includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or Kk-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Muller & Roland E. Kontermann, Bispecific Antibodies, HANDBOOK OF THERAPEUTIC ANTIBODIES 265-310 (2014), the entirety of which is herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein, the term “multispecific antibody” refers to an antibody with binding specificities for at least two different antigens. While such molecules normally will only bind two antigens (e.g., bispecific antibodies, bsAbs), antibodies with additional specificities such as trispecific antibody and KIH Trispecific are also contemplated.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies can be prepared using a wide variety of techniques known in the art including the use of hybridoma, recombinant, and phage display technologies, or a combination thereof.

The term “dsRNA binding protein” refers to a protein with a dsRNA binding domain. A dsRNA binding protein may include an RNA silencing suppressor protein. A “RNA silencing suppressor protein” refers to a protein that are encoded by viruses to suppress host RNA silencing. In an exemplary embodiment, the virus is an insect virus, such as a Flock house virus (FHV). A “FHV” refers to an insect virus of the Nodaviridae family. In an exemplary embodiment, the RNA silencing suppressor protein is an FHV B2 protein. The “FHV B2 protein” or “B2 protein” is a homodimer that indiscriminately binds to double-stranded RNA (dsRNA) molecules independent of their nucleotide sequences and sizes such as siRNAs duplexes and long dsRNAs, thereby protecting dsRNA from being accessed and processed by dicer2 of the RNAi machinery.

In recent years, mRNA-based vaccines have emerged as a versatile platform for rapid and targeted development of vaccines against infectious diseases. Due to the success of mRNA-based vaccines, significant efforts have been dedicated to evaluating mRNA technologies for a broad range of potential applications, such as enzyme replacement, and anti-viral therapies.

The broad range of possible therapeutic uses for mRNAs has been made possible through the development of in vitro transcribed (IVT) mRNA. IVT mRNA is a single-stranded transcript that has been engineered to mimic naturally occurring mRNAs in the cytoplasm of eukaryotic cell. In vitro transcription is generally performed with a T7 RNA polymerase in the presence of nucleotides. However, due to the nature of T7 RNA polymerase, transcription abortion or slippage can occur, which can produce either short or long dsRNA byproducts.

Upon entering human cells, dsRNA impurities can trigger an innate immune and inflammatory response by: (1) activating dsRNA-dependent enzymes, such as oligoadenylated synthetase (OAS), RNA-activated protein kinase (PKR), and RNA-specific adenosine deaminase (ADAR), causing inhibition of protein translation; and (2) stimulating endogenous dsRNA sensors, including Toll-like receptor 3 (TLR3), melanoma differentiation-associated protein 5 (MDA5) and retinoic acid-inducible gene I (RIG-I), resulting in the activation of innate immunity and secretion of various cytokines, e.g. interleukin-6 (IL-6), tumor necrosis factor-α and type I interferons. Accordingly, dsRNA impurities have the potential to affect the potency and safety of mRNA drug products.

Approaches to reduce the effect of dsRNA in an mRNA drug product include: (1) replacing uridine in mRNA with modified uridines such as m1ψ or 5moU, (2) optimizing IVT reaction condition or T7 polymerase to reduce dsRNA formation, and (3) subjecting IVT mRNA to further purification. To ensure the success of these approaches, it is critical to monitor the level of dsRNA in IVT mRNA products and confirm the removal of dsRNA after purification.

Several methods for detecting and quantifying dsRNA include: dot-blot assay, enzyme-linked immunosorbent assay (ELISA), homogeneous time resolve fluorescence (HTRF) assay, and bioluminescence assay (e.g., Promega Lumit® assay). Lateral flow immunoassay (LFSA) may be used for for dsRNA quantitation (see, e.g., Dengwang Luo et al., Molecular Therapy Nucleic Acids, 2023, 32, 445-453). These assays may utilize one or more antibodies that target dsRNA, polyinosinic:polycytidylic acid (poly(I:C)), or another synthetic analog of dsRNA. These antibodies may include J2, J5, and K1. Although these antibodies have been used extensively for more than 30 years, little is known about their binding affinity towards dsRNA with modified uridines.

J2, J5, and K1 may have differences in binding affinity, and in particular, the K1 antibody was shown to have weaker binding affinity towards dsRNA (see, e.g., Markus Baiersdorfer et al., Molecular Therapy Nucleic Acids, 2019, 15, 26-35). Indeed, a combination of K1 and J5 antibodies used in LFSA showed reduced detection sensitivity for dsRNA with m1ψmodification, which is a common modification in mRNA-based therapeutics or vaccines. Therefore, conventional methods (e.g., conventional ELISA assays or assays using conventional antibodies) may be unable to reliably report dsRNA levels in nucleic acids with modified RNA (e.g., modified uracil).

Flock House Virus (FHV) is an insect virus which includes a positive sense RNA genome. In insect host cells, an RNA silencing pathway may be utilized as a self-defense mechanism against viral infection. The RNA silencing pathway depends on the formation of RNA-inducing silencing complex (RISC), which targets siRNA derived from FHV genome to specifically degrade the viral genome. It was discovered that the FHV genome encodes a protein called B2, which acts as a countermeasure against RNA silencing (Jeffery A Chao et al., Nature Structural and Molecular Biology, 2005, 12, 952-957). The suppression mechanism of RNA silencing by B2 has been shown to operate in dual mode: (1) by binding to the double strand RNA formed during viral genome replication, which can block cleavage activity by DICER, an RNase enzyme, and (2) by binding to siRNA generated by DICER cleavage and sequestering its incorporation into the RISC.

The B2 protein is a potent dsRNA binder which can bind to dsRNA as short as 17 bp, and has a binding mode different from canonical double-stranded RNA-binding domains (dsRBD). Further, multiple B2 proteins may bind to one dsRNA at the same time, with stoichiometry increasing accordingly to the length of dsRNA (Chao et al., 2005). Due to its ability to bind long and short dsRNA in a sequence independent manner, B2 has been utilized to study dsRNA in living cells, including but not limited to dsRNA location and distribution (Baptiste Monison et al., Frontiers in Plant Science, 2018, 9, 70 and Xiaofei Cheng et al., Virology, 2015, 485, 439-451).

Biolayer interferometry (BLI) is an optical technique capable of rapid and robust titer/concentration measurement and quantification of various biologics, including but not limited to antibodies, adeno association virus (AAV) and host cell protein (HCP). BLI has various practical advantages, including rapid read-out, simple operation, and low requirements for sample process. Additionally, the versatility of BLI extends to measuring binding affinity for various RNA-protein interactions.

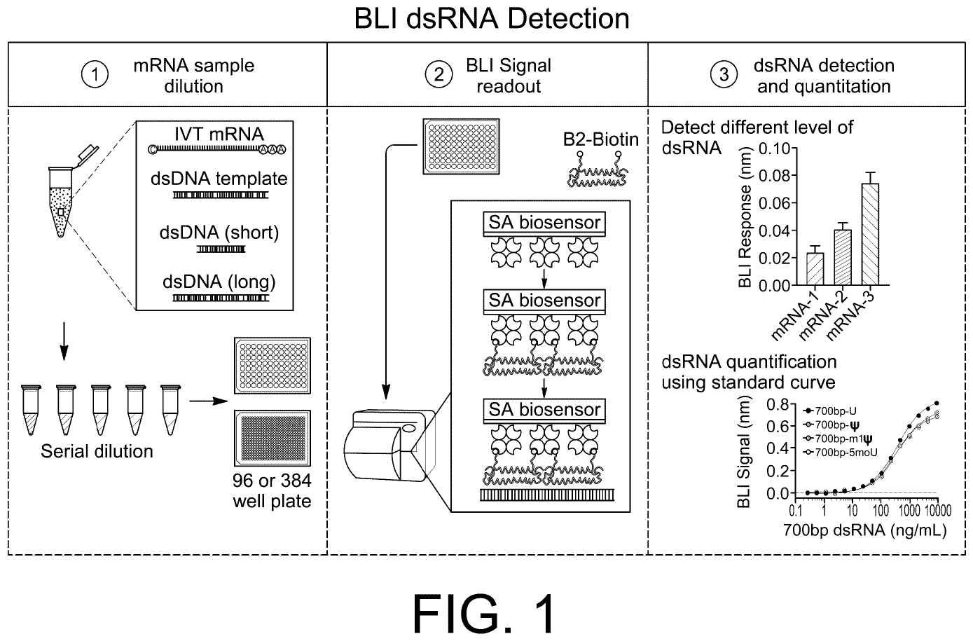

In the present disclosure, the binding affinity of Flock House Virus (FHV) B2 protein, J2 antibody, and K1 antibody for modified and unmodified dsRNA was investigated using BLI. Following this, a method for detecting dsRNA was developed, which leveraged the specificity of the B2 protein towards dsRNA and the straightforward application of BLI (FIG. 1). Altogether, the BLI dsRNA detection method enables rapid and quantitative monitoring of dsRNA in IVT mRNA products, regardless of nucleoside modification, and can be easily implemented as release testing for mRNA drug product manufacturing.

In an exemplary embodiment, a method is provided for detecting and quantifying dsRNA in a sample using biolayer interferometry (BLI). As used herein, the term “biolayer interferometry (BLI)” refers to a label-free technique for measuring biomolecular interactions. BLI is an optical analysis technique that analyzes the interference pattern of white light reflected from two surfaces of an inner interference layer and an immobilized capture molecule layer on the biosensor tip. Any change in the number of molecules bound to the biosensor tip causes a shift in the interference pattern that can be measured in real time (Yasmina N Abdiche et al., Analytical Biochemistry, 2008, 377(2), 209-217).

It is understood that the present invention is not limited to any of the aforesaid nucleic acids(s), capture molecule(s), proteins(s), antibody(ies), and biolayer interferometry, and any nucleic acids(s), capture molecule(s), proteins(s), antibody(ies), or biolayer interferometry can be selected by any suitable means.

### EXAMPLES

Reagents. 1 M Tris, pH 8.0, 5 M NaCl, and yeast tRNA (RNase free grade) were purchased from ThermoFisher Scientific (Waltham, MA). Tween™ 20 was also purchased from Thermofisher Scientific (Waltham, MA).

Recombinant B2 protein construct design, expression, and purification. Residue 1-73 of FHV B2 protein (Uniprot P68831) were cloned into a pET30a vector. Two different constructs of B2 were generated: one with N-terminal 6×His tag (referred to as B2) and another with N-terminal 6×His tag and C-terminal GS linker follower by an AviTag (referred to as B2-Avi). The two constructs were expressed by E. coli BL21(DE3) and a two-step purification (Ni column+Superdex 75 column) method was employed for the protein purification. For expression of B2-Avi, a BirA plasmid was co-transfected with the B2 plasmid to enable co-translational biotinylation of B2-Avi protein (referred to as B2-Biotin). Protein identity was confirmed by SDS-PAGE and LC-MS (data not shown). All vector design, construct, protein expression and purification were performed by Genscript (Piscataway, NJ).

Preparation of dsRNA standards. A list of dsRNA standards of different length and uridine modifications were analyzed in this study (Table 1). These dsRNA standards were prepared by annealing complementary ssRNA strands produced from IVT. A hairpin dsRNA which contains a 100A poly(A) tail and 70 bp complimentary sequence before and after the poly(A) tail, which mimics the dsRNA byproduct from IVT, was also included in the study. The listed dsRNA standards were prepared by CATUG (CATUG Biotechnology, Suzhou, China).

TABLE 1
List of dsRNA standards used in this study.

| | | Estimated | | |
|---|---|---|---|---|
| | | dsRNA | | M.W. |
| dsRNA | Description | length^^{∧} | | (kDa) |
| 25 bp-U | Short dsRNA*, uridine | 25 | bp | 16.0 |
| 25 bp-m1ψ | Short dsRNA*, m1ψ | 25 | bp | 16.1 |
| 700 bp-U | dsRNA based on GLuc mRNA, | 557 | bp | 367.6 |
| | uridine | | | |
| 700 bp-m1ψ | dsRNA based on GLuc mRNA, m1ψ | 557 | bp | 371.4 |
| 700 bp-ψ | dsRNA based on GLuc mRNA, ψ | 557 | bp | 367.6 |
| 700 bp-5moU | dsRNA based on GLuc mRNA, | 557 | bp | 378.2 |
| | 5moU | | | |
| 1800 bp-U | dsRNA based on FLuc mRNA, | 1652 | bp | 1090.3 |
| | uridine | | | |
| 1800 bp-m1ψ | dsRNA based on FLuc mRNA, m1ψ | 1652 | bp | 1098.7 |
| Hairpin-U | 642-nt mRNA with poly(A) tail | 70 | bp | 291.7 |
| | 70-bp complementary strand before | | | |
| | and after poly(A), uridine | | | |
| Hairpin-m1ψ | 642-nt mRNA with poly(A) tail | 70 | bp | 293.4 |
| | 70-bp complementary strand before | | | |
| | and after poly(A), m1ψ | | | |

*25 bp dsRNA sequence as follows: (sense) 5′ CACGUACCGUUCUAGCGGGCUCUCG 3′ (SEQ ID NO: 1); (anti-sense) 3′ GUGCAUGGCAAGAUCGCCCGAGAGC 5′ (SEQ ID NO: 2).
^{∧}Estimated based on sequence complementarity.

The purity of the dsRNA standards was confirmed by capillary electrophoresis (CE) (Table 2). The double strand content for these dsRNA standards were confirmed by HTRF assay and ELISA.

TABLE 2
dsRNA purity analysis by capillary electrophoresis. The corrected
peak area % (CPA %) values are an average of duplicate runs.

| dsRNA | Purity |
|---|---|
| samples | (CPA %) |
| 25 bp-U | 89.4 |
| 25 bp-m1ψ | 85.4 |
| 700 bp-U | 96.5 |
| 700 bp-m1ψ | 98.1 |
| 700 bp-ψ | 99.2 |
| 700 bp-5moU | 91.8 |
| 1800 bp-U | 96.6 |
| 1800 bp-m1ψ | 94.5 |
| Hairpin-U | 87.3 |
| Hairpin-m1ψ | 84.6 |

IVT mRNA preparation. PCR products generated from plasmid templates (Genscript USA, Piscataway, NJ) or digested linearized plasmid were used as the template for mRNA. mRNAs were generated by in vitro transcription using a customized ribonucleoside blend of Cleancap AG (3′Ome) (Trilink Biotechnologies, San Diego, CA), guanosine triphosphate, adenosine triphosphate, cytidine triphosphate (Thermofisher Scientific, Waltham, MA), and N1-Methyl-Pseudouridine-5′-Triphosphate (m1ψ) (TriLink Biotechnologies, San Diego, CA) and T7 polymerase (Thermofisher Scientific, Waltham, MA). The mRNA product was purified with the Tangential flow filtration or silica beads. The purified mRNA was then quantified using a NanoDrop spectrometer (Thermofisher Scientific, Waltham, MA) or a Qubit (Thermofisher Scientific, Waltham, MA).

sRNA and dsDNA preparation. ssRNA (100-nt) was chemically synthesized by Integrated DNA Technologies (Coralville, IA). dsDNA was prepared from plasmid template using PCR. Briefly, PCRs were performed using a KAPA HiFi HotStart ReadyMix (Roche, Indianapolis, IN). The linear DNA generated from PCR was then purified by QIAquick PCR purification kit (QIAGEN, Inc., Redwood City, CA), and the purity was confirmed by 1% Invitrogen E-Gel Agarose Gel using an E-Gel™ Power Snap Electrophoresis System (Thermo Fisher Scientific, Waltham, MA) (data not shown).

Capillary electrophoresis (CE) of dsRNA purity. A PA800 Plus system (Sciex, Framingham, MA) equipped with a bare fused-silica capillary (30.2 cm) with an inner diameter of 50 m was used for CE analysis. 1.5% Polyvinyl-pyrrolidone (Millipore Sigma, St. Louis, MO) with 1×TBE (Millipore Sigma, St. Louis, MO) was used as a separation buffer. The ladder was prepared by adding 99 μL of nuclease-free water to 1 μL of a mixture of dsRNA Ladder (NEB, Ipswich, MA). The samples were diluted to 5 ng/mL in nuclease-free water. 90 L of the prepared sample from each mixture was transferred to a sample vial for injection. The sample chamber temperature was set to 10° C. with the capillary temperature at 30° C., and the sample was injected under a voltage of 3 kV for 3 s with reversed polarity. Electrophoretic separation was carried out with a voltage of 6 kV with reversed polarity for 22 min. The detection window was localized 10.2 cm from the outlet of the capillary to ensure an effective separation length of 20 cm. Laser-induced fluorescence detection was used to monitor the separation with an excitation wavelength of 488 nm and an emission wavelength of 520 nm.

Homogenous time resolved fluorescence (HTRF) assay. HTRF was performed using a viral dsRNA detection kit (Revvity, Waltham, MA). The provided standard (5 μg/mL) was prepared in triplicates at 100 ng/mL and then serially diluted to 7 concentrations. The samples (dsRNA or mRNA) were diluted within the linear range of the standard curve. 10 μL of each sample and standard was added to a small volume detection white microplate. The detection antibodies (Eu Cryptate and d2) were diluted 50-fold in 1×detection buffer, mixed at a ratio of 1:1, and 10 μL of the mixture was added to each well of the microplate. The plate was then sealed and incubated overnight at 4° C., and the acceptor and donor emission signals at 665 nm and 620 nm, respectively, was read the following day. A ratio was calculated from the acceptor and donor emission signals to produce a standard curve and to quantify the level of dsRNA in the samples.

ELISA with J2 antibody ELISA was performed using an ELISA kit (Exalpha, Shirley, MA). The ELISA plate was coated with J2 antibody diluted in PBS and incubated at 4° C. overnight. After coating with the J2 antibody, the plate was blocked using a blocking buffer (1% BSA in PBS+0.2% NaN3) for two hours. After washing of the plate, 100 μL of poly (I:C) dsRNA Positive Control (30 ng dsRNA/well starting and serially diluted 1:3 for 11 concentrations and one blank) and diluted dsRNA or mRNA sample (prepared in STE Buffer: 0.1 M NaCl, 1 mM EDTA, 50 mM Tris-HCl, pH 7.0) were added to each well. The plate was incubated for one hour.

Subsequently, undiluted K1 antibody was added to each well of the plate. The plate was incubated for one hour before 100 μL of diluted secondary antibody (HRP-conjugated F(ab)2 Fragment of goat-anti mouse; 1.3 μL of antibody to 21 mL PBS+1% BSA) was added to each well. After incubation of the plate for one hour, TMB (3,3′,5,5′ tetramethylbenzidine) ELISA substrate was added to each well and the plate was incubated for 5-60 minutes in the dark while absorbance at 650 nm was read every minute using a BioTek Synergy Neo2 Reader (Shoreline, WA). When absorbance at 650 nm reached the optimum level, the reaction was stopped and the absorbance at 450 nm was read using a BioTek Synergy Neo2 Reader (Shoreline, WA).

Biolayer interferometry (BLI) method for binding affinity measurements. Binding affinity was measured using the Octet® RED384 (Sartorius). Briefly, streptavidin biosensors (SA biosensors, Sartorius) were first hydrolyzed and equilibrated with assay buffer (10 mM Tris, pH 8.0, 100 mM NaCl, 0.1 mg/mL tRNA, 0.1 mM EDTA, 0.05% Tween-20) for at least 20 minutes. B2-Biotin protein was diluted to 5 μg/mL in assay buffer (10 mM Tris, pH 8.0, 100 mM NaCl, 0.1 mg/mL tRNA, 0.1 mM EDTA, 0.05% Tween-20), and was captured on SA biosensors to a density of >4 molecules per nm² to create a dense surface for avidity binding to dsRNA. The B2 immobilized surface was then used to capture dsRNA to a level of about 0.3 molecules per nm². Binding responses were generated by serial titration of B2 (1017 nM to 16 nM for dsRNA without modification and 2543 nM to 40 nM for all modified dsRNA) to dsRNA surface with a blank control. Steady state analysis (Octet® BLI Analysis v12.2 and Graphpad Prism) was used for the binding affinity determination.

BLI for dsRNA detection. The dsRNA detection assay was developed using the Octet® RED384 (Sartorius). The streptavidin biosensors were prepared the same as described above for binding affinity measurements. The dsRNA standard curves were generated by serial titration of dsRNA (4 μg/mL to 0.24 ng/mL) with a blank control. The level of dsRNA in an IVT mRNA product was determined from a binding response generated by serial titration of the mRNA sample (40 to 5 μg/mL) to the B2 surface.

Specificity and interference testing of BLI dsRNA detection assay. For specificity testing, 20 μg/mL of ssRNA and dsDNA were tested using the BLI dsRNA detection assay, together with 0.2 μg/mL of 700 bp dsRNA (U or m1ψ), 142 bp dsRNA (Jena Biosciences) and Poly(I:C) as positive control (Sigma-Aldrich). The assay was performed the same as described above for dsRNA detection assay. For interference testing, 0.2 ug/mL of 700 bp dsRNA (U or m1ψ) was mixed with 100-fold or 200-fold (w/w) of ssRNA or dsDNA. The mixture was tested using the BLI dsRNA detection assay as described above.

Ion pair reverse phase FPLC purification. Reverse phase purification for IVT mRNA samples was performed on AKTA Pure (Cytiva, Marlorough, MA) using a RNAsep semi-preparative column (21.2 mm×100 mm, ADS Biotec, Catalog number RPC-99-2110, Omaha, NE) with mobile phase A (MPA, ADS Biotec, Omaha, NE): 0.1 M triethylamine acetate (TEAA) pH 7.0 and mobile phase B (MPB, ADS Biotec, Omaha, NE): 0.1 M TEAA, 25% acetonitrile, pH 7.0. The IVT mRNA samples were purified with a linear gradient from 38% to 60% MPB in 6 column volumes (CVs). Fractions containing purified mRNA were buffer exchanged using nuclear free water (Thermofisher Scientific, Waltham, MA) to remove the organic solvents.

### Example 1

Both short and long dsRNA impurities have been found as by-products of in vitro transcription of mRNA. Thus, in order to detect dsRNA impurities, the B2 protein must be able to bind short and long dsRNA with high affinity. The binding affinity of B2 protein for short dsRNA has been characterized using electrophoretic mobility shift assay (EMSA) (Chao et al., 2005). In particular, they demonstrated that B2 displays similar high affinity for dsRNA between 17 and 25 bp in length (Chao et al., 2005). Although multiple studies have utilized B2 for in vitro or in vivo study of dsRNA, there is little report on the binding affinity of B2 for long dsRNA, possibly due to the aggregation of complex formed between B2 and longer dsRNA.

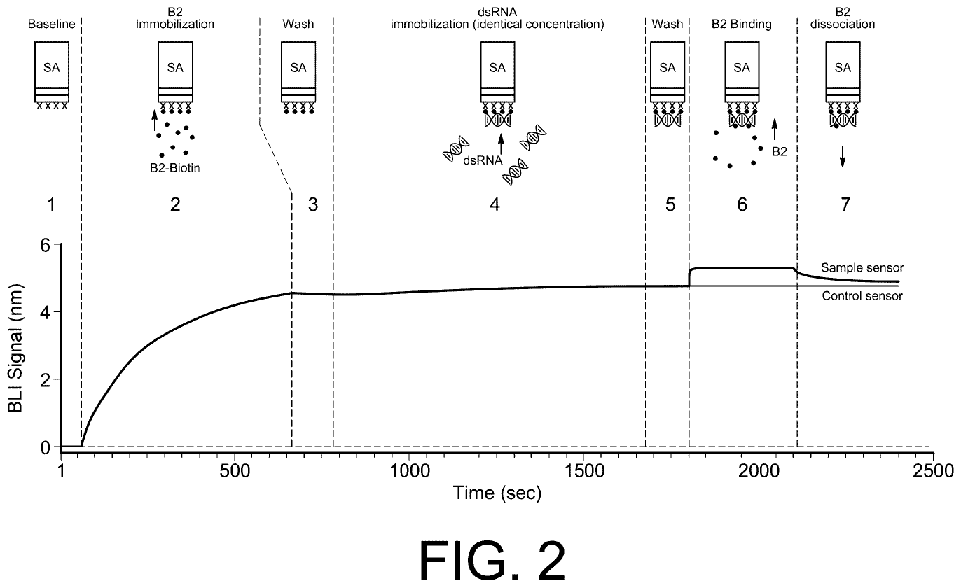

As part of characterizing the B2 protein, a BLI method was developed to measure the binding affinity of B2 for long and short dsRNAs (FIG. 2). As used herein, long dsRNA may refer to RNA molecules having a double-stranded region that comprises at least 25 base pairs, such as, for example, at least 50 base pairs, at least 100 base pairs, at least 150 base pairs, at least 200 base pairs, at least 250 base pairs, at least 300 base pairs, at least 400 base pairs, at least 500 base pairs, at least 600 base pairs, at least 700 base pairs, at least 800 base pairs, at least 900 base pairs, at least 1000 base pairs, at least 1500 base pairs, at least 2000 base pairs, at least 3000 base pairs, at least 4000 base pairs, at least 5000 base pairs, at least 6000 base pairs, at least 7000 base pairs, at least 8000 base pairs, or at least 9000 base pairs. The BLI method for affinity measurement may comprise: (1) loading B2-biotin onto streptavidin biosensors; (2) capturing dsRNA on B2 loaded biosensors; and/or (3) measuring the binding response of serial diluted B2 to the dsRNA loaded biosensors. As shown in FIG. 2, once captured by B2, the immobilized dsRNA surface is very stable, thus providing a stable baseline for affinity measurement. The immobilization of dsRNA on the biosensor allows each binding site on the dsRNA to be treated independently, which enables use of a 1:1 binding model for binding affinity measurement.

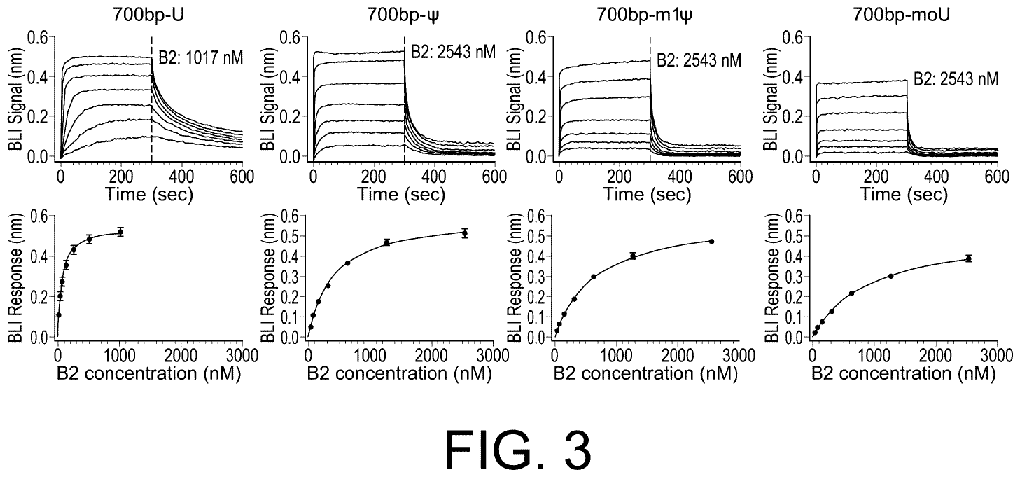

Using the methods described herein, the binding affinity of B2 for 700 bp-U dsRNA was measured (FIG. 3 and Table 3). Due to the fast on-rate of the binding kinetics, steady state analysis was used to determine the K_{D} value and the standard error of mean (SEM) from three independent replicates. The average BLI signals, i.e. the binding response, from 290 to 295 seconds were plotted against B2 concentration. From this analysis, the K_{D} value for B2 for 700 bp-U was determined to be −60 nM. This high affinity binding is consistent with a previous finding where B2 was shown to bind to long dsRNA; however, in this previous study, binding affinity was not determined (Monison et al., 2015).

TABLE 3
Binding affinity of B2 for different 700 bp dsRNA standards.

| dsRNA binder | dsRNA | K_{D} (nM) | Rₘₐₓ (nm) |
|---|---|---|---|
| B2 | 700 bp-U | 62 ± 4 | 0.545 ± 0.010 |
| | 700 bp-ψ | 405 ± 20 | 0.604 ± 0.010 |
| | 700 bp-m1ψ | 669 ± 26 | 0.605 ± 0.009 |
| | 700 bp-5moU | 948 ± 44 | 0.531 ± 0.011 |

### Example 2

To investigate whether chemical modifications to uridine in long dsRNA has an impact on B2-dsRNA interaction, the binding affinities for a set of 700 bp dsRNA with different uridine modifications, pseudouridine (W), N1-methylpseudouridine (m1ψ), and 5-methoxyuridine (5moU), were measured. Comparison of each modifications' binding response suggested that interactions between 700 bp-U and B2 was the strongest, followed by 700 bp-ψ, 700 bp-mly, and 700 bp-5moU, with 700 bp-5moU being the weakest (FIG. 3). Overall, there was more than a 15-fold difference in the K_{D} value between 700 bp-U and 700 bp-5moU, as summarized in Table 3. One possible explanation for the weakened binding of uridine-modified dsRNA to B2 is that uridine modifications can affect base-pairing interactions, which can prevent the formation of dsRNA.

The same BLI method was also adapted to measure the binding affinity of two commonly used anti-dsRNA antibodies, J2 and K1, to dsRNA with or without modified uridines. Comparison of the measured K_{D} values suggested that J2 showed a stronger binding affinity towards dsRNA than K1 (Tables 4 and 5). Similar to B2, uridine modification impacted J2 (or K1)—dsRNA interaction. Overall, there was at least a 4-fold difference in the binding affinity of J2 (or K1) to 700 bp-U and 700 bp-5moU (Tables 4 and 5).

The impact of uridine modification on the binding affinity of the J2 antibody followed the same trend as B2, but with a more modest difference (5-fold vs. 15-fold for 5moU, for example). It is worth noting that the K_{D} value determined for J2 is the overall affinity from the two Fab arms, and not the affinity from each individual Fab arm, which explains the modest differences in binding affinity due to the avidity effect.

The K1 antibody had a weaker binding affinity for unmodified and modified dsRNA than J2 antibody (Tables 4 and 5). This is consistent with earlier qualitative reports that K1 antibody has weaker interactions with dsRNA (Jürgen W Schönborn et al., Nucleic Acids Research, 1991, 19, 2993-3000, Luo et al., 2023, and Monison et al, 2015.). Further, a recent study found that when a combination of K1 (capture) and J5 (detection) antibodies is used, the detection sensitivity for m1ψdsRNA is worse than that for uridine-containing dsRNA (Luo et al., 2023). This observation may be explained in part by the reduced affinity of K1 antibody for m1ψdsRNA, as demonstrated in this study.

TABLE 4
J2 binding affinity towards dsRNA with different uridine modification.
K_{D}, kₒₙ and k_{off} mean and standard error of mean (SEM) values
were obtained from three independent replicates.

| dsRNA | | | | |
|---|---|---|---|---|
| binder | dsRNA | K_{D} (nM) | kₒₙ (1/Ms) | k_{off} (1/s) |
| J2 | 700 bp-U | 27 ± 3 | (2.11 ± 0.21) E⁵ | (5.65 ± 0.09) E⁻³ |
| | 700 bp-ψ | 32 ± 5 | (1.89 ± 0.50) E⁵ | (6.03 ± 1.05) E⁻³ |
| | 700 bp-m1ψ | 56 ± 1 | (1.22 ± 0.02) E⁵ | (6.80 ± 0.18) E⁻³ |
| | 700 bp- | 131 ± 5 | (8.68 ± 0.51) E⁴ | (1.14 ± 0.08) E⁻² |
| | 5moU | | | |

TABLE 5
K1 binding affinity towards dsRNA with different uridine
modification. K_{D} mean and standard error of mean (SEM)
values were obtained from three independent replicates.

| dsRNA binder | dsRNA | K_{D} (nM) |
|---|---|---|
| K1 | 700 bp-U | 325 ± 33 |
| | 700 bp-ψ | |
| | 700 bp-m1ψ | μM range |
| | 700 bp-5moU | |

### Example 3

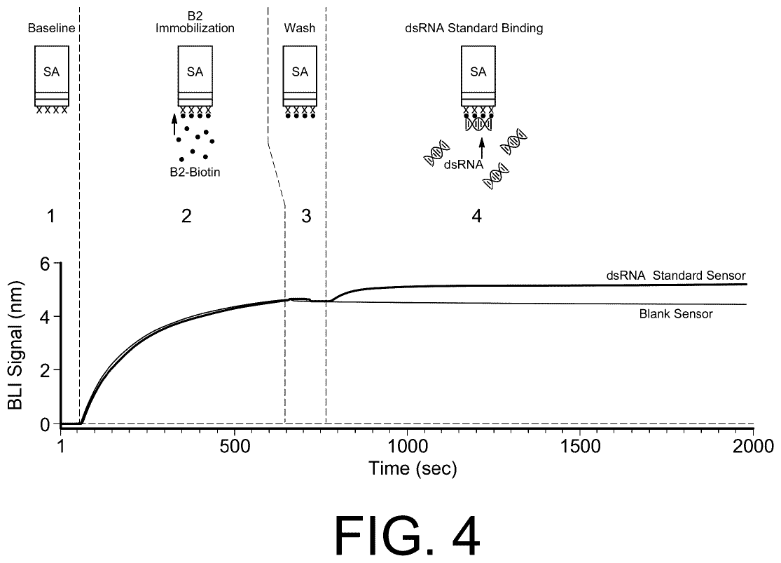

The BLI dsRNA detection assay is advantageous in that it requires a relatively few amount of steps, compared to conventional detection assays. The BLI dsRNA detection assay may include: (1) immobilization of B2-biotin on the SA biosensors to create a dense surface of B2 protein to enable avidity of binding; and/or (2) dipping of the B2-immobilized biosensors into a sample to detect the presence of dsRNA (FIG. 4). The level of dsRNA is reflected by the binding response upon binding of the B2 protein to dsRNA. Notably, the total run time of the BLI method is less than 35 minutes (per 16 biosensors).

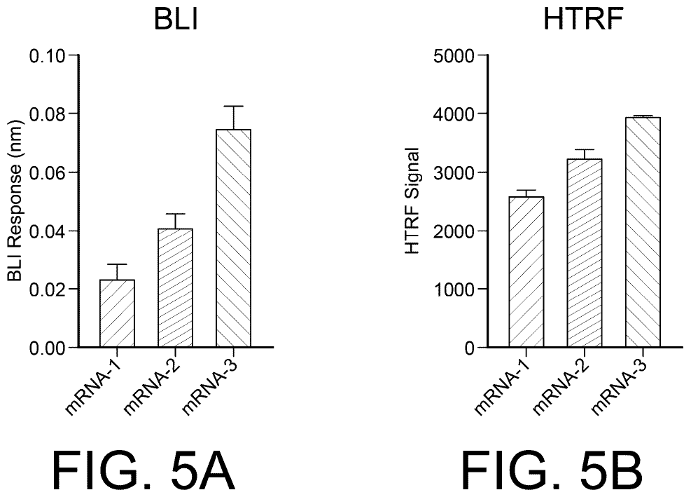

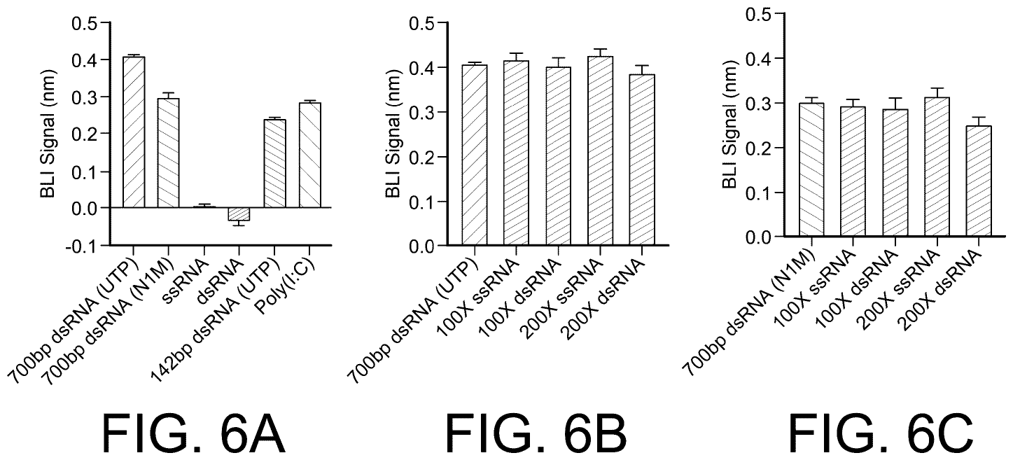

To validate the feasibility of the BLI dsRNA detection assay, the BLI responses of three IVT mRNA samples, mRNA-1, mRNA-2, and mRNA-3 were compared with the signals from the HTRF assay (FIG. 5). Based on BLI responses, the level of dsRNA was highest in mRNA-3, followed by mRNA-2 and mRNA-1. These results were consistent with results from HTRF analysis, which showed the same trend in dsRNA levels for the three mRNA samples. Together, these results suggest that BLI dsRNA detection assay can reliably detect dsRNA levels in IVT mRNA samples.

### Example 4

Methods described herein for detecting dsRNA demonstrate high specificity in addition to high sensitivity. In vitro transcription of dsRNA often results in dsDNA (linearized template) and ssRNA byproducts. These two types of nucleic acids share a similar chemical composition to that of dsRNA, and may also be more abundant than dsRNA impurities. Therefore, the ability of the BLI dsRNA detection assay to distinguish dsRNA from dsDNA and ssRNA was evaluated.

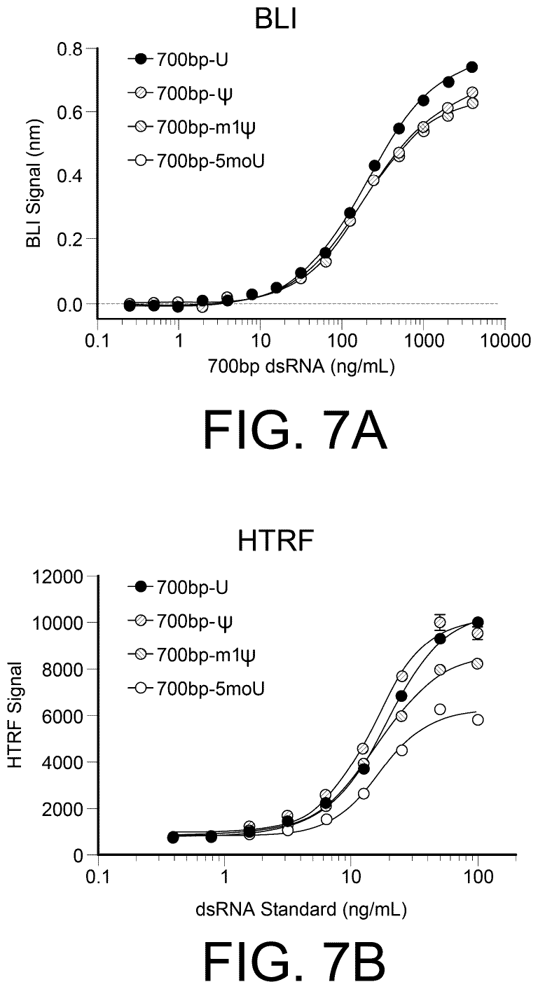

Three dsRNA samples, including 700 bp dsRNA (U or mly), 142 bp dsRNA, and Poly(I:C) (positive control), were tested in parallel with dsDNA and ssRNA at a concentration100-fold than dsRNA. Minimal or even negative BLI responses were observed for samples containing 100-fold more ssRNA and dsDNA than dsRNA (20 μg/mL vs 0.2 μg/mL) (FIG. 6A). In contrast, the dsRNA and positive control samples clearly displayed a signal (FIG. 6A). Taken together, these results suggest that the BLI detection method has little cross-reactivity with ssRNA or dsDNA.

Further, the impact of ssRNA or dsDNA on dsRNA signal was examined. Two dsRNA samples, 700 bp-U or 700 bp-m1ψ(concentration of 0.2 μg/mL), were tested separately using the BLI dsRNA detection assay in the presence of increasing amounts of ssRNA or dsDNA (FIGS. 6B and 6C). There was no statistically significant difference in BLI responses between samples with or without ssRNA or dsDNA, indicating that excess ssRNA or dsDNA has minimal interference in the BLI dsRNA detection assay.

### Example 5

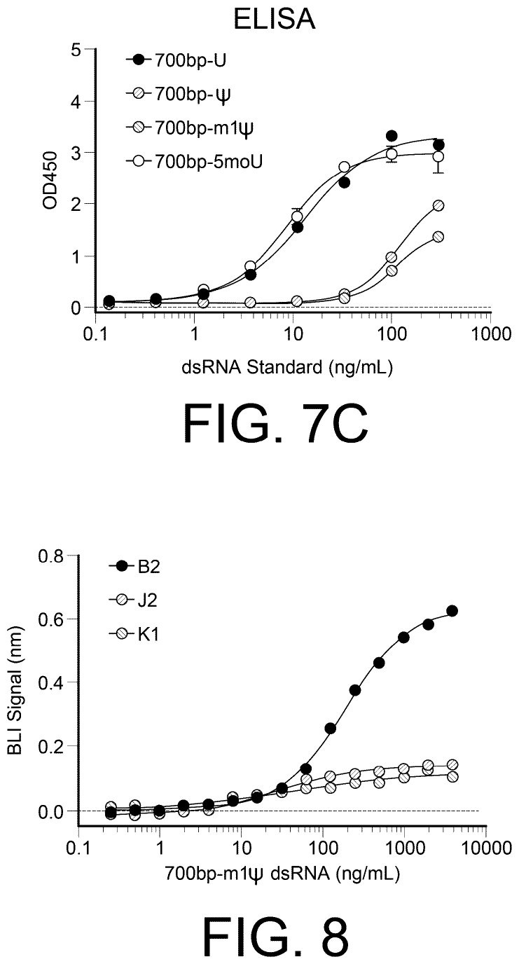

For quantitative analysis of dsRNA using the BLI dsRNA detection method, a standard curve must be generated using dsRNA standards with known concentrations. To generate the standard curve, BLI response was measured at a given time frame for various concentrations of dsRNA with different uridine modifications, and the BLI signal was plotted against dsRNA standard concentration. Similar to the standard curves generated by HTRF or ELISA, the standard curves for each dsRNA adopted a S-shape response (FIG. 7A). The standard curves were then fitted using a 4-parameter logistic (4PL) model. The lower limit of detection (LOD) was determined based on a 3:1 signal-to-noise ratio (S/N) and the lower limit of quantitation (LOQ) was determined based on a 10:1 S/N, where noise represents the average responses from blank samples.

To understand whether avidity effect (or the dense immobilization for B2 on biosensors) may achieve similar detection sensitivity for dsRNA with or without uridine modifications, standard curves generated for 700 bp-ψ, 700 bp-m1ψ and 700 bp-5moU were compared with the standard curve of 700 bp-U (FIG. 7). Compared to 700 bp-U, standard curves of 700 bp-m1ψ and 700 bp-5moU showed a lower high asymptote but similar EC50 values (Table 6). The LOD and LOQ values obtained for the modified dsRNA were similar to the unmodified dsRNA (Table 7).

Together, these results show that the BLI dsRNA detection assay exhibits similar detection sensitivity for unmodified and modified dsRNA.

As comparison, the same set of 700 bp dsRNA standards (U, ψ, m1ψ and 5moU) was tested on a HTRF assay and ELISA using the J2 antibody (J2 ELISA). With HTRF, standard curves for 700 bp-m1ψ and 700 bp-5moU showed roughly 20% and 40% lower high asymptote, respectively, compared to 700 bp-U (FIG. 7B). It is worth noting that HTRF does not use J2 or K1 antibodies, and instead uses a mouse antibody isolated in 1980s (Yoshichika Kitagawa et al., Analytical Biochemistry, 1981, 115, 102-108). This implies that the weaker binding affinity towards m1ψ or 5moU modified dsRNA might be a general property of anti-dsRNA antibodies. With J2 ELISA, standard curves for m1ψ and W modified dsRNA showed a considerable right-shift, indicating reduced detection sensitivity that could potentially cause more than 10-fold under-reporting of modified dsRNA impurities (FIG. 7C).

The difference in binding affinity for modified and unmodified dsRNA could impact the detection sensitivity of an assay. For current commercially available dsRNA quantitation assays, such as HTRF and ELISA, the dsRNA standards used for quantitation is pre-made and does not include uridine modification. The use of generic standards can result in an underestimation of the levels of dsRNA impurity in mRNA products with uridine modification.

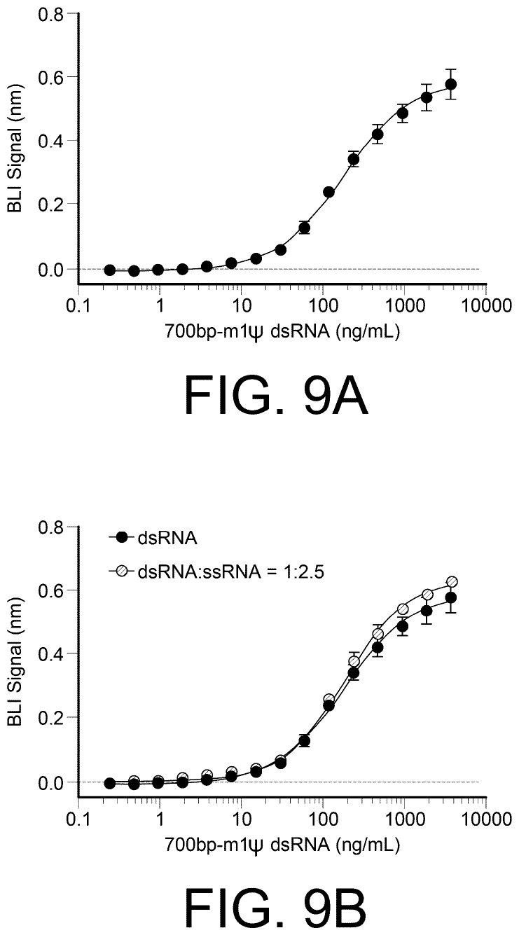

In contrast to HTRF and ELISA, the BLI dsRNA detection method can overcome differences in binding affinity for modified and unmodified dsRNA. In this method, a biosensor surface immobilized with a saturating amount of B2 greatly reduced the impact of the weaker binding, resulting in similar detection sensitivities for dsRNA with or without uridine modification (FIG. 6A and Table 7). Interestingly, in the BLI dsRNA detection assay, a reduced dynamic range (signal between high asymptote and low asymptote) was observed when the B2 protein was replaced with J2 or K1 antibody (FIG. 8). This result highlights the importance of a dense capture surface for quantitation.

TABLE 6
Representative fitting parameters for dsRNA with various uridine
modifications obtained using the BLI dsRNA detection assay.

| dsRNA | Bottom (nm) | Top (nm) | Hillslope | EC50 (ng/mL) |
|---|---|---|---|---|
| 700 bp-U | −0.006067 | 0.7703 | 1.023 | 204.6 |
| 700 bp-m1ψ | 0.002437 | 0.6377 | 1.110 | 191.7 |
| 700 bp-5moU | −0.009762 | 0.6802 | 0.965 | 211.0 |
| 700 bp-ψ | −0.001121 | 0.6843 | 0.972 | 204.8 |

TABLE 7
Limits of detection and limit of quantitation for different dsRNA
standards determined for BLI dsRNA detection method. The LoD
is defined as the lowest dsRNA concentration giving a signal
greater than the nonspecific binding (mean of six or twelve
measurements of BLI assay buffer) + 3x standard deviations
(six or twelve measurements). The LoQ is defined as the lowest
dsRNA concentration giving a signal greater than the nonspecific
binding (mean of six or twelve measurements of BLI assay buffer)
+ 10x standard deviations (six or twelve measurements).

| | | Limit of detection | Limit of quantitation |
|---|---|---|---|
| dsRNA binder | dsRNA | (ng/mL) | (ng/mL) |
| B2 | 700 bp-U | 14.0 | 59.5 |
| | 700 bp-ψ | 13.7 | 64.6 |
| | 700 bp-m1ψ | 27.9 | 89.7 |
| | 700 bp-5moU | 15.9 | 70.0 |
| | 25 bp-U | 9.3 | 41.2 |
| | 25 bp-m1ψ | 21.5 | 154.6 |
| | 1800 bp-U | 23.8 | 74.0 |
| | 1800 bp-m1ψ | 35.2 | 94.0 |
| | Hairpin-U | 17.4 | 58.6 |
| | Hairpin-m1ψ | 16.1 | 84.0 |

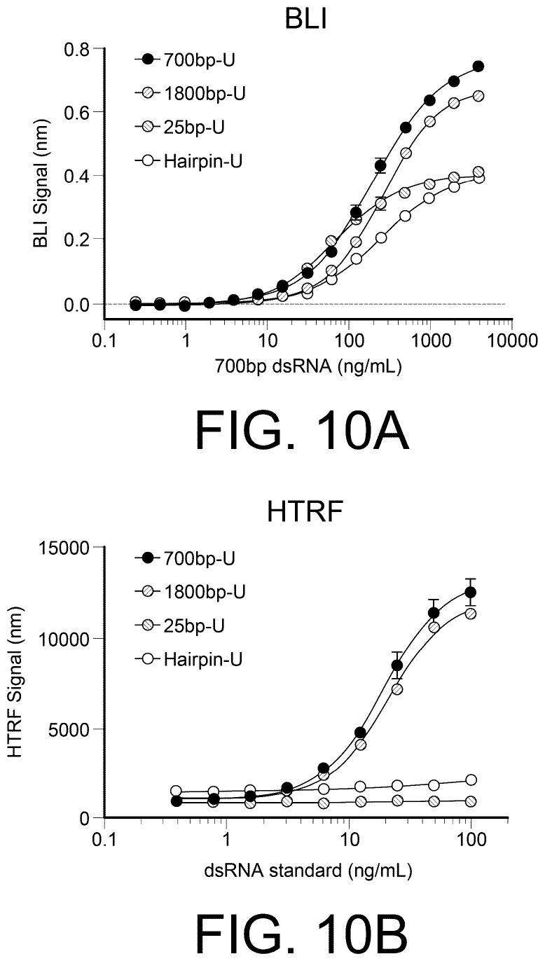

To test whether the presence of excess ssRNA would impact sensitivity, standard curves for 700 bp-m1ψdsRNA in the presence of 2.5-fold of ssRNA was generated (FIGS. 9A and 9B). No major difference was observed when comparing the standard curves in the presence and absence of ssRNA (FIG. 7B), which suggests that the presence of excessive amount of ssRNA has a minimal impact on the quantitation of dsRNA by the BLI dsRNA method.

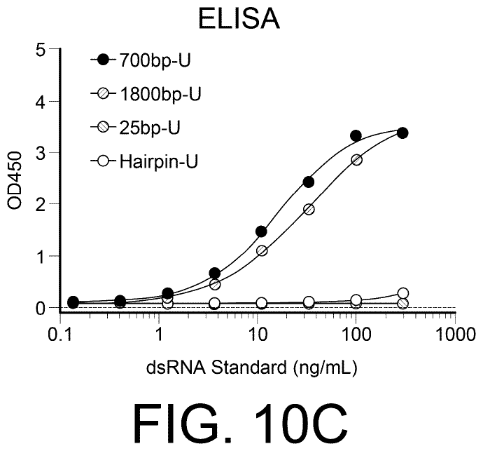

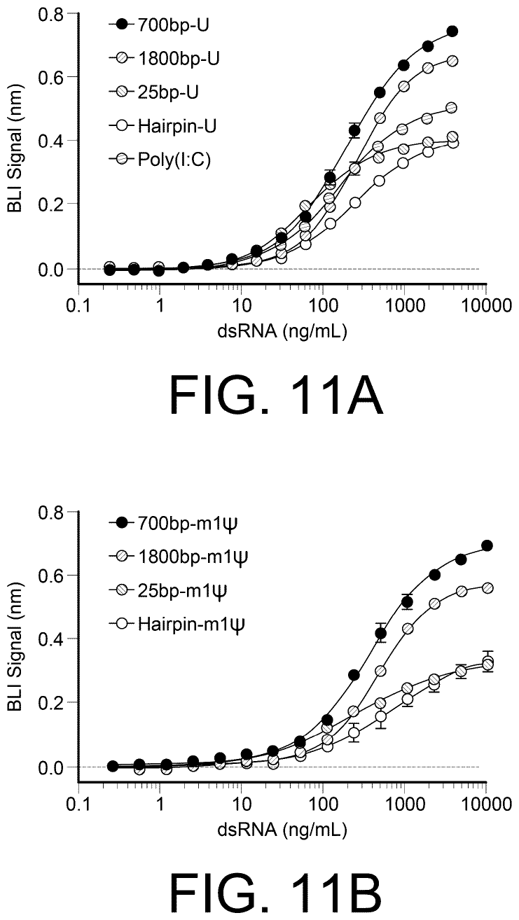

To test whether the BLI dsRNA detection assay can quantify dsRNA impurities of different lengths and structural features, standard curves for 25 bp dsRNA (U or m1ψ), 1800 bp dsRNA (U or m1ψ) and hairpin dsRNA (U or m1ψ) were generated (FIG. 10A and FIG. 11). The length of the 25 bp dsRNA is close to the shortest dsRNA that B2 has been shown to bind (17 bp). Compared to the standard curve generated for the long dsRNA, the short dsRNA showed a lower high asymptote (FIG. 10A and FIG. 11). However, comparable sensitivity was observed between short and long dsRNA based on the determined LOD and LOQ values (Table 7). This suggests that the BLI dsRNA detection assay can detect and quantify dsRNA as short as 25 bp (U or m1ψ). In contrast, the general recognized length limit for J2 or K1 antibody-based detection is 40 bp. Indeed, HTRF assay or J2 ELISA detected minimal signal when 25 bp dsRNA standards were used (FIGS. 10B and 10C).

Further, comparable sensitivity was observed between hairpin dsRNA and duplex dsRNA (Table 7), as evidenced by the standard curves generated with the hairpin dsRNA (FIG. 8). Unlike the 25 bp and 1800 bp dsRNAs samples, which have largely a duplex structure, hairpin dsRNA contains a dsRNA loop that is formed by complementary regions before and after poly(A) tail. The hairpin dsRNA sample mimics the loop-back dsRNA impurities generated by IVT. This loop back structure was detected by the BLI dsRNA detection assay, but it was not detected by HTRF or J2 ELISA (FIGS. 10A-10C). Thus, this finding indicates that BLI dsRNA detection assay offers increased detection sensitivity for hairpin dsRNA. Together, these results suggest that the BLI dsRNA detection assay may provide a more complete picture of dsRNA impurities in IVT mRNA.

### Example 6

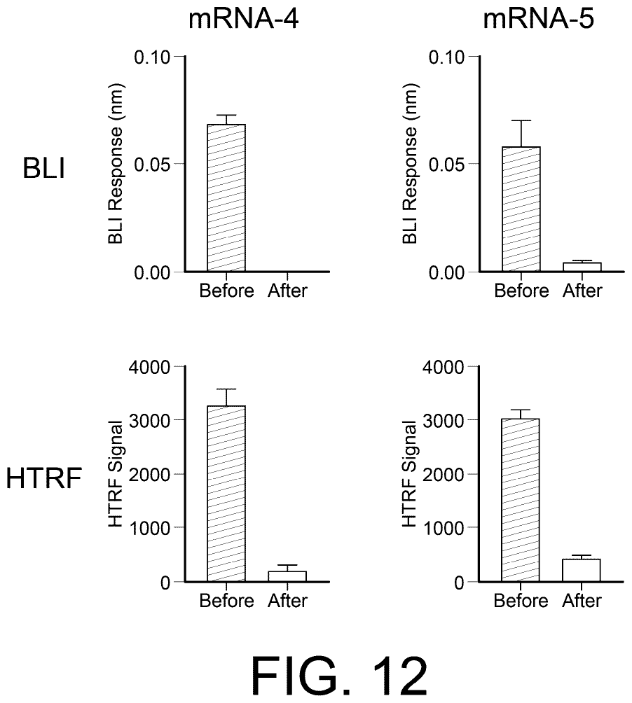

Ion pair reverse phase (IPRP) purification has been demonstrated as a reliable way of removing dsRNA impurities from IVT mRNA and reduce immunogenicity (Katalin Karikó et al., Nucleic Acids Research, 2011, 39, e142). To test whether the BLI dsRNA detection assay can be used to monitor dsRNA clearance after IPRP purification, two IVT mRNA samples were subjected to IPRP purification and the dsRNA levels of the two samples before and after IPRP purification were measured using the BLI dsRNA detection assay and HTRF assay. As expected, a nearly 100% reduction in dsRNA level was observed for both mRNA-4 and mRNA-5 following IPRP purification as shown by BLI assay (FIG. 12). A similar reduction in dsRNA levels was observed by the HTRF assay (FIG. 12). This confirms that the BLI dsRNA detection assay can be used to monitor dsRNA impurities removal through purification process.

The present disclosure provides a novel BLI dsRNA detection assay which can rapidly and reliably detect dsRNA impurities in IVT mRNA. The binding affinities of B2 for dsRNA of uridine modification were systematically determined using the BLI dsRNA detection assay. Interestingly, binding affinity was inversely related to the degree of modification on the uridine. dsRNA with 5moU had the most significant modification and highest K_{D} (i.e., weakest binding affinity) while W has the least modification in terms of chemical changes and lowest K_{D} i.e., strongest binding affinity).

In addition to B2, two commonly used anti-dsRNA antibodies, namely J2 and K1, which are critical antibody reagents in various ELISA kits for dsRNA quantitation, also showed weaker binding towards dsRNA with modifications. Although isolated more than 30 years ago, this is the first study, to our knowledge, that measured the K_{D} values of J2 and K1 for dsRNA with or without modifications. For J2 antibody, the impact of uridine modification on binding affinity followed the same trend as B2, but with a more modest difference (5-fold vs. 15-fold for 5moU, for example). This difference in binding affinity for modified and unmodified dsRNA could impact the sensitivity of commercial assays which use unmodified dsRNA standards to measure modified dsRNA impurities.

To overcome the effects of uridine modification of B2 on dsRNA interaction while maintaining an easy-to-implement assay, the binding avidity of the B2 protein was leveraged in the BLI detection assay to mitigate the effects of reduced binding affinity of B2. In this design, a biosensor surface is immobilized with a saturating amount of B2, which greatly reduces the impact of the weaker binding and results in similar detection sensitivities for dsRNA with or without uridine modification. If accurate quantitation is the goal, given the difference in the high asymptote of the standard curves generated using dsRNA with different uridine modification, it is still recommended to use dsRNA standard with the same uridine modification as used in the IVT process for producing mRNA.

Lastly, it was demonstrated that this BLI dsRNA detection assay can successfully monitor dsRNA removal. Given the fast run time of this assay (35 minutes), it could be an ideal at-line test to implement for monitoring dsRNA impurities between process steps. Further, given that Octet® instruments are 21CFR part 11 compliant, this assay could be qualified as release testing as well.

The present disclosure is further described by the following non-limiting items.

Item 1. A method for detecting a double-stranded RNA (dsRNA) in a sample, comprising:
(a) immobilizing a capture molecule to a solid surface;
(b) contacting a sample including the dsRNA to the solid surface, wherein the capture molecule immobilized on the solid surface specifically binds to the dsRNA; and
(c) measuring a binding response of the dsRNA to the solid surface using biolayer interferometry.

Item 2. The item of item 1, wherein the dsRNA is an impurity of a mRNA product produced by in vitro transcription.

Item 3. The method of item 1, wherein the dsRNA is between about 25 bp to about 7000 bp.

Item 4. The method of item 1, wherein the dsRNA is between about 700 bp to about 1800 bp.

Item 5. The method of item 1, wherein the dsRNA comprises a modified nucleoside.

Item 6. The method of item 5, wherein the nucleoside is uridine.

Item 7. The method of item 5, wherein the modified nucleoside comprises pseudouridine (W), N1-methylpseudouridine (m1ψ) or 5-methoxyuridine (5moU).

Item 8. The method of item 1, wherein the dsRNA comprises a duplex structure.

Item 9. The method of item 1, wherein the dsRNA comprises a hairpin loop.

Item 10. The method of item 1, wherein the dsRNA is present in the sample at a concentration of at least 5 ng/mL.

Item 11. The method of item 1, wherein the sample further comprises double-stranded DNA (dsDNA) and/or single-stranded RNA (ssRNA).

Item 12. The method of item 11, wherein the concentration of the dsDNA or ssRNA is greater than the concentration of the dsRNA.

Item 13. The method of item 11, wherein the concentration of the dsDNA or ssRNA is more than a 100-fold greater than the concentration of the dsRNA.

Item 14. The method of item 1, wherein the capture molecule is a dsRNA binding protein.

Item 15. The method of item 14, wherein the dsRNA binding protein is a Flock House Virus (FHV) B2 protein.

Item 16. The method of item 1, wherein the capture molecule is selected from a group consisting of an antibody, a receptor, a fragment thereof, and a combination thereof.

Item 17. The method of item 16, wherein the antibody is a J2 antibody, a K1 antibody, or a J5 antibody.

Item 18. The method of item 1, wherein the dsRNA is bound to one or more capture molecules.

Item 19. The method of item 1, wherein immobilizing comprises contacting a biotinylated capture molecule to a solid surface that is coated with avidin, streptavidin, or a variant thereof.

Item 20. The method of item 1, wherein the solid surface is immobilized with a saturating amount of capture molecule.

Item 21. The method of item 1, wherein the solid surface has a capture molecule density of greater than 4 nm.

Item 22. The method of item 1, wherein the solid surface is washed to remove unbound capture molecule prior to contacting the sample to the solid surface.

Item 23. A method for quantifying dsRNA in a sample, comprising:
(a) immobilizing a capture molecule to a solid surface;
(b) contacting a sample including the dsRNA to the solid surface, wherein the capture molecule immobilized on the solid surface binds to the dsRNA;
(c) measuring a binding response of the dsRNA to the solid surface using biolayer interferometry; and
(d) comparing the binding response to a standard curve relating binding response to concentration to determine a concentration of the dsRNA in the sample.

Item 24. The method of item 23, wherein the dsRNA is an impurity of a mRNA product produced by in vitro transcription.

Item 25. The method of item 23, wherein the dsRNA is between about 25 bp to about 7000 bp.

Item 26. The method of item 23, wherein the dsRNA is between about 700 bp to about 1800 bp.

Item 27. The method of item 23, wherein the dsRNA comprises a modified nucleoside.

Item 28. The method of item 27, wherein the nucleoside is uridine.

Item 29. The method of item 27, wherein the modified nucleoside comprises pseudouridine (W), N1-methylpseudouridine (m1ψ) or 5-methoxyuridine (5moU).

Item 30. The method of item 23, wherein the dsRNA comprises a duplex structure.

Item 31. The method of item 23, wherein the dsRNA comprises a hairpin loop.

Item 32. The method of item 23, wherein the dsRNA is present in the sample at a concentration of at least 40 ng/mL.

Item 33. The method of item 23, wherein the sample further comprises double-stranded DNA (dsDNA) and/or single-stranded RNA (ssRNA).

Item 34. The method of item 33, wherein the concentration of the dsDNA or ssRNA is greater than the concentration of the dsRNA.

Item 35. The method of item 33, wherein the concentration of the dsDNA or ssRNA is more than a 100-fold greater than the concentration of the dsRNA.

Item 36. The method of item 23, wherein the capture molecule is a dsRNA binding protein.

Item 37. The method of item 36, wherein the dsRNA binding protein is a Flock House Virus (FHV) B2 protein.

Item 38. The method of item 23, wherein the capture molecule is selected from a group consisting of an antibody, a receptor, a fragment thereof, and a combination thereof.

Item 39. The method of item 38, wherein the antibody is a J2 antibody, a K1 antibody, or a J5 antibody.

Item 40. The method of item 23, wherein the dsRNA is bound to one or more capture molecules

Item 41. The method of item 23, wherein immobilizing comprises contacting a biotinylated capture molecule to a solid surface that is coated with avidin, streptavidin, or a variant thereof.

Item 42. The method of item 23, wherein the solid surface is immobilized with a saturating amount of capture molecule.

Item 43. The method of item 23, wherein the solid surface has a capture molecule density of greater than 4 nm.

Item 44. The method of item 23, wherein the standard curve is fitted to a 4-parameter logistic model.

Item 45. The method of item 23, wherein the solid surface is washed to remove unbound capture molecule prior to contacting the sample to the solid surface.

All references cited herein, including U.S. patent and applications are incorporated by reference in their entirety. The present disclosure is not to be limited in terms to the particular examples described in this application, which are intended as illustrations of various aspects. Many modifications and variations can be made without departing from its spirit and scope, as will be apparent to those skilled in the art. Functionally equivalent methods within the scope of the disclosure, in addition to those enumerated herein, will be apparent to those skilled in the art from the foregoing descriptions. Such modifications and variations are intended to fall within the scope of the appended claims.

What is claimed is:

1. A method for detecting double-stranded RNA (dsRNA) in a sample, the method comprising:
immobilizing a capture molecule to a solid support, wherein the capture molecule specifically binds to dsRNA;
contacting a sample including dsRNA to the solid support; and
measuring a binding response of the dsRNA to the capture molecule using biolayer interferometry.

2. The method of claim 1, wherein the dsRNA comprises a modified nucleoside.

3. The method of claim 1, wherein the dsRNA comprises pseudouridine, N1-methylpseudouridine, and/or 5-methoxyuridine.

4. The method of claim 1, wherein the dsRNA comprises a duplex structure or a hairpin loop.

5. The method of claim 1, wherein the dsRNA is present in the sample at a concentration of about 5 ng/mL to about 5000 ng/mL.

6. The method of claim 1, wherein a concentration of double-stranded DNA (dsDNA) in the sample is greater than a concentration of dsRNA in the sample; and/or
wherein a concentration of single-stranded RNA (ssRNA) in the sample is greater than a concentration of dsRNA in the sample.

7. The method of claim 1, wherein the capture molecule is an antibody, a receptor, an antibody fragment, a receptor fragment, or a combination thereof.

8. The method of claim 1, wherein the capture molecule includes a J2 antibody, a K1 antibody, a J5 antibody, a Flock House Virus (FHV) B2 protein, or a combination thereof.

9. The method of claim 1, wherein immobilizing the capture molecule to the solid support comprises contacting a biotinylated capture molecule to a surface of the solid support, wherein the surface comprises avidin, streptavidin, or a variant thereof.

10. The method of claim 1, wherein immobilizing the capture molecule to the solid support comprises immobilizing the capture molecule at a density of at least about 4 molecules per square nanometer.

11. A method for quantifying dsRNA in a sample, the method comprising:
immobilizing a capture molecule to a solid support, wherein the capture molecule binds to dsRNA;
contacting a sample including dsRNA to the solid support;
measuring a binding response of the dsRNA to the capture molecule using biolayer interferometry; and
comparing the binding response to a standard curve relating binding response to concentration to determine a concentration of the dsRNA in the sample.

12. The method of claim 11, wherein the sample is a product of mRNA in vitro transcription.

13. The method of claim 11, wherein the dsRNA comprises a modified nucleoside.

14. The method of claim 11, wherein the sample further comprises double-stranded DNA and/or single-stranded RNA.

15. The method of claim 11, wherein a concentration of double-stranded DNA in the sample is more than 100-fold greater than the concentration of dsRNA in the sample; and/or
a concentration of single-stranded RNA in the sample is more than 100-fold greater than the concentration of dsRNA in the sample.

16. The method of claim 11, further comprising, after immobilizing the capture molecule and prior to contacting the sample to the solid support, washing the solid support, thereby removing unbound capture molecule.

17. The method of claim 11, wherein contacting the sample to the solid support causes a dsRNA molecule within the sample to bind to one or more capture molecules.

18. The method of claim 11, wherein the standard curve is fit to a 4-parameter logistic model.

19. The method of claim 11, wherein the dsRNA comprises, a hairpin loop, pseudouridine, N1-methylpseudouridine, and/or 5-methoxyuridine.

20. The method of claim 11, wherein immobilizing the capture molecule to the solid support comprises contacting a biotinylated capture molecule to a surface of the solid support, wherein the surface comprises avidin, streptavidin, or a variant thereof, such that the capture molecule is present on the surface at a density of at least about 4 molecules per square nanometer.


SEQUENCE LISTING (2 sequences)

SEQ ID NO: 1 - RNA, 25 nt, Synthetic construct
  source 1..25: mol_type = other RNA
cacgtaccgttctagcgggctctcg

SEQ ID NO: 2 - RNA, 25 nt, Synthetic construct
  source 1..25: mol_type = other RNA
cgagagcccgctagaacggtacgtg